(19) United States
(12) Patent Application Publication
(10) Pub. No.: US 2008/0015475 A1
(43) Pub. Date: Jan. 17, 2008

(54) Manual resuscitation device

(75) Inventors: Greg Y. Lau, Mooresville, NC (US); Christopher B. Reed, Mocksville, NC (US); Douglas B. Constable, Bermuda Run, NC (US); Robert Martin, Mocksville, NC (US)

Correspondence Address: Gilbert J. Andia, Jr., Suite 500, 101 W. Friendly Ave, Greensboro, NC, 27415, US

(73) Assignee: Ventlab Corporation

(21) Appl. No.: 11/484,896

(22) Filed: Jul. 12, 2006

(51) Int. Cl. A61H 31/00; A61M 16/00

(52) U.S. Cl. 601/41; 128/205.13

(57) ABSTRACT

An improved cardiopulmonary resuscitation device for resuscitating human infants is provided. The device includes a manually compressible and resiliently expandable bag constructed of an elastomer shell that encloses a breathable gas chamber having a volume capacity sufficient to inflate the lungs of a human patient when the bag is fully compressed. The shell includes at least one compressible region constructed of a wall having less resilience than the shell's average resilience. The less resilient region helps prevent over inflation of the patient's lungs. The device further includes a patient interface assembly that is communicable with a breathable gas outlet of the bag breathable gas chamber, and a breathable gas source communicable with an inlet of the bag breathable gas chamber. The device can optionally include a breathable gas manometer to monitor backpressure from the patient's lungs to further insure against over inflating the patient's lungs.

### BACKGROUND OF THE INVENTION

(1) Field of the Invention

The present invention relates to a disposable cardiopulmonary resuscitation device. In particular, the present invention is directed to an improved cardiopulmonary resuscitation bag that helps prevent the inadvertent over inflation of delicate lungs such as those belonging to a human infant.

(2) Description of the Prior Art

A disposable cardiopulmonary resuscitation (CPR) device is used to externally assist the breathing of a patient whose natural breathing ability is dangerously diminished. This device is also commonly called a Bag-Valve-Mask (BVM) for the three major components of the device, or a CPR bag, or a manual resuscitator. Generally, a CPR or BVM device incorporates some type of manually compressible and resilient bulb or bag having a breathable air chamber that forces air through a patient interface such as a facial mask when the bag is manually squeezed. The resiliency of the bag allows for the bag to refill quickly and automatically. Most modern CPR devices include at least two one-way valves; one valve that conducts the flow of breathable gas from the bag's breathable gas chamber into a patient valve; and, the second that allows the bag to refill during expansion of the resilient bulb. The patient valve allows the flow of air to be conducted to a patient interface, such as a mask or endotracheal tube, and provides an exhaust port that directs the patient's lung exhaust away from the resilient bulb to the atmosphere.

The advantage of these manual resuscitators over electromechanical assisted breathing devices is that CPR devices can be used by first responders in the field where electromechanical devices would be cumbersome or inoperable. Moreover, CPR devices are quick to implement and are relatively simple in construction, making them inherently reliable.

Manual resuscitators are employed in an assisted breathing session by interfacing the patient to the device. In most cases, this action involves a rescue person placing and sealing the CPR device's facemask over the nose and mouth of the patient. Next, the rescue person manually compresses the compressible and self-restoring bag to force air from the breathable gas chamber and into patient's lungs. As the rescue person releases the compression of the bag, the resiliency of the bag restores the breathable gas chamber to its uncompressed volume. The patient's inflated lungs naturally expel the patient's exhaust gases, and the valve directs the exhaust flow to the atmosphere. The rescue person alternately compresses and releases the CPR bag at a natural breathing frequency until the patient regains their natural breathing ability or until advanced medical respiration equipment is made available, such as when the patient reaches a hospital or other such equipped trauma center.

While the performance of modern CPR devices is good, the possibility of inadvertent over inflation of a patient's lung resulting in lung damage is ever present. Lung over inflation is of particular concern whenever a CPR device is used to resuscitate infants.

Another method of administering oxygen to assist the respirations of a patient is commonly called an anesthesia bag or hyperinflation bag. An anesthesia bag has a reservoir or bag that is not self inflating, and thus a continuous source of gas must be available and connected to the device. The anesthesia bag is a balloon type device, typically made of latex, whose first end is connected to a patient interface (mask or endotracheal tube or the like) through an elbow connection. The second end of the bag is connected to a compressed gas or oxygen supply. The anesthesia bag does not typically require a valve. The anesthesia bag fills with oxygen and remains inflated as oxygen is provided to the patient. If the patient needs an additional influx of oxygen, the bag may be squeezed directing the stored oxygen into the valve and to the patient, allowing the lungs to by hyperinflated.

Anesthesia bags allow for slow and gentle ventilations to the patient without excessively high peak airway pressures that can be delivered with CPR bags. Many operators tend to briskly squeeze the stiffer CPR bag due to the poorer “feel” of the bag, which generates higher peak airflows. Anesthesia bags are often thin walled latex that provide greater feedback to the medical professional, allowing the medical professional to know the level of inflation that is needed to the lungs, and to prevent overinflation. The more supple bag of the anesthesia bag device is less fatiguing to the hands of the user during prolonged use.

Prior art attempts to address the problem of excessive peak airflow and overinflation of a patient have included the addition of manometers to the CPR device to allow the rescue person to monitor the pressure applied to the patient's lungs during compression. An example of one such manometer is disclosed by U.S. Pat. No. 5,557,049 to Ratner. Although the addition of manometers to CPR devices is an improvement, this addition does not address the issue of inexperienced rescue persons over inflating a patient's lungs in a stressful environment in which the rescue person may be distracted from monitoring the manometer. As a result of distractions, the rescue person may inadvertently ignore the manometer and over inflate the patient's lungs. Moreover, the manometer might not be visible in all environments in which resuscitation is being performed. Therefore, what is needed is a CPR device that prevents a rescue person from over inflating a patient's lungs regardless of rescue environment, experience or stress.

### SUMMARY OF THE INVENTION

The present invention addresses the need by providing a manual resuscitator that greatly reduces the chances that a rescue person will over inflate a patient's lungs with or without the use of a lung pressure manometer. Generally, the cardiopulmonary resuscitation device of the present invention is comprised of a manually compressible and resiliently expandable self-restoring bag that is constructed of an elastomer shell having one end with a breathable gas inlet and an opposite end having a breathable gas outlet. A breathable gas chamber within the elastomer shell has a volume capacity sufficient to inflate the lungs of a human patient when the bag's shell is fully compressed. A person of skill in the art will know that the size of the chamber can be varied for devices designed for children, infants and/or new born children.

The resuscitator of the present invention includes a compressible bag having at least one thin walled area having a wall thickness that is thinner than the remainder of the compressible bag. This at least one thin walled area has less resilience than the shell's average resilience, and attempts to provide the feel and feedback that a clinician would get from an anesthesia bag. The at least one thin walled area is sized to comfortably match a portion of the clinician's hand while not affecting the ability of the bag to reinflate or to provide sufficient volume of air to the patient.

It is preferred that the bag's shell have two such defined thin walled areas, one for the thumb, and one for a finger (or fingers) of the rescuer's bag compressing hand. Moreover, it is preferable for the shell to include tactile indicators to help the rescuer locate the thumb and forefinger soft spot compressible regions in low visibility circumstances. In one embodiment, the tactile indicator for at least one thin walled area is a circular detent having a diameter of approximately 1.5 inches. Other tactile indicators include, but are not limited to, bumps and/or ridges.

A thin walled area having suitable characteristics for the present invention is preferably constructed from the same elastomer material as the rest of the bulb. It is preferable that the bag be compression molded or injection molded with liquid silicone. In this way, all thin walled regions can be easily manufactured integral with the bulb. However, in order to achieve a desirable resiliency that is substantially less than the average resiliency of the shell, the wall thickness of a suitable thin walled region needs to be less than the wall thickness of the shell overall. It is preferred that the average wall thickness of the bag is approximately 2.5 mm. It is preferred that the thin walled region have a wall thickness of less than 0.5 mm. Other ways to produce a desirable resiliency that is substantially less than the average resiliency of the shell can be, but is not limited to, substituting another material of lesser resilience in place of the overall shell material, and/or reducing the resilience controlling properties of the shell material at the locations of the soft spot compressible regions being manufactured.

The resuscitator of the present invention preferably includes a patient interface such as a facial mask that is in communication with the breathable gas outlet of the bag's shell. It is preferred for the patient interface to be connected to a patient valve assembly that has an inlet port connected to the breathable gas outlet of the bag's shell. The patient valve assembly may also include a gas manometer such as disclosed by the Ratner patent referred to in the prior art description. The patient valve is typically designed to provide for minimum resistance to the passage of the breathable gas from the bag into the patient interface, while providing a mechanism for preventing exhaled gases from entering into the bag and preferably being vented to atmosphere.

The resuscitator of the present invention can optionally include a medical oxygen source having a medical gas line that is a conduit between the medical gas source and the breathable gas inlet of the bag's shell. Furthermore, the CPR device can optionally include an oxygen collection bladder to collect oxygen from the medical oxygen source before it is mixed with ambient air inside the shell's breathable gas chamber.

In operation of the CPR device of the present invention, a rescue person will first interface the patient with the bag device. The interface in almost all instances will be a facemask. Therefore, the rescuer will need to establish that the facemask has a sufficient seal around the mouth and nose of the patient. The rescuer will grasp the compressible bulb of the manual resuscitator with at least one hand, and orient that hand so that the thumb and one or more fingers are placed over the thin walled area of the bag. The rescuer can compress the bulb by squeezing on the thin walled areas, forcing breathable gas into the patient's lungs. Once sufficiently compressed to deliver appropriate volume to the patient, the rescuer will release the bag to allow it to restore under its own resiliency, thereby pulling more breathable gas through the shell's inlet and into the breathable gas chamber. The rescuer will alternately compress and release the bag's shell in like fashion at a normal breathing rate until the patient's breathing is restored or until advanced breathing equipment is made available to the patient. If the device is optionally equipped with an oxygen source, line and collection bladder, the rescuer will turn on the oxygen source to enrich the breathable gas oxygen content.

Other aspects of the present invention will become apparent to one skilled in the art upon a reading of the following detailed description of the invention, taken with the drawings.

### BRIEF DESCRIPTION OF THE DRAWINGS

FIG. 1, is a top view of the cardiopulmonary manual resuscitation device showing a cut-away view of the manually compressible and resiliently expandable bag.

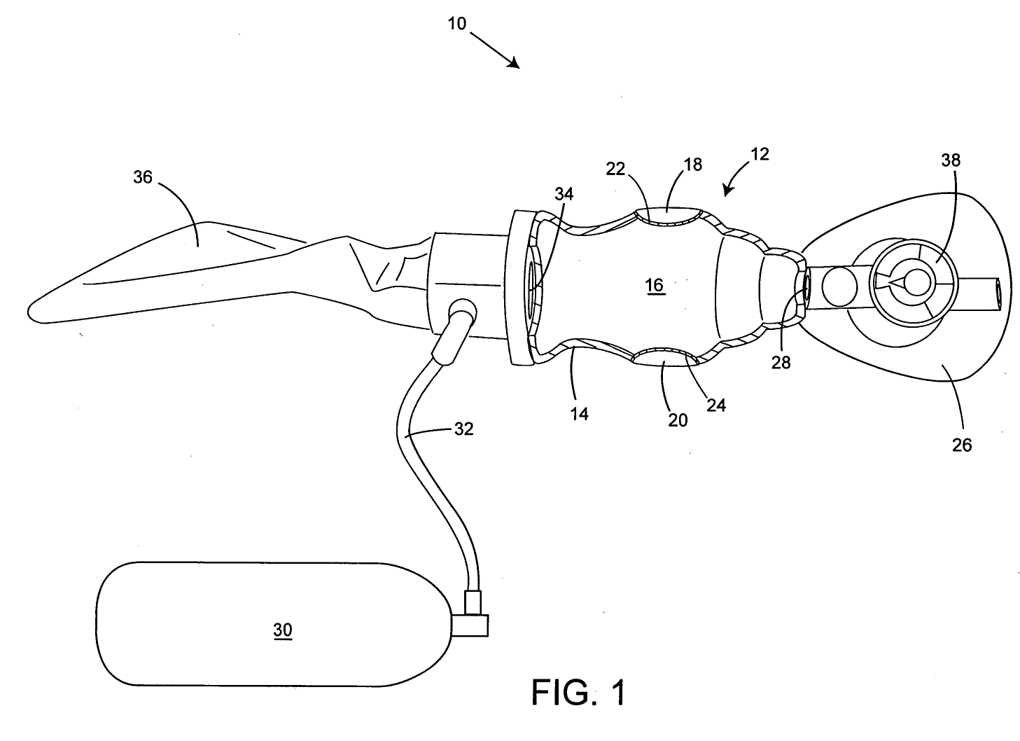

FIG. 2, is a side view of the cardiopulmonary manual resuscitation device.

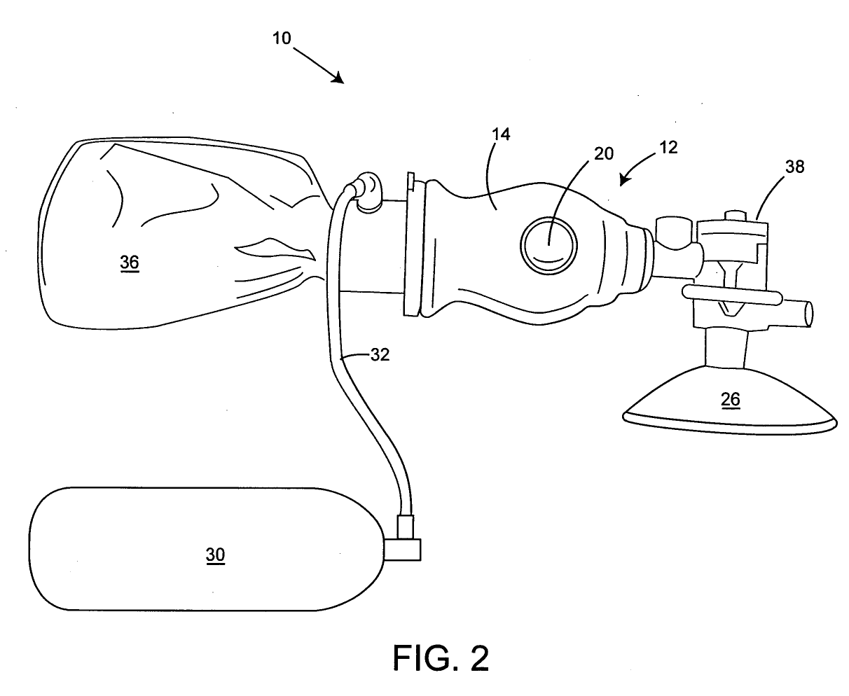

### DETAILED DESCRIPTION OF THE PREFERRED EMBODIMENT

The resuscitation device of the present invention, generally 10, is comprised of a manually compressible and resiliently expandable bag 12 that is constructed of an elastomer shell 14 having a generally uniform thickness. A breathable gas chamber 16 has a volume capacity to inflate the lungs of a human patient when bag 12 is sufficiently compressed. Bag 12 is preferably manufactured in a range of sizes to fully inflate the lungs of infants, children and adults.

Shell 14 includes at least one integral thin-walled compressible region and preferably two integral thin-walled compressible regions 18 and 20. These regions each have a wall, 22 and 24 respectively. The walls 22 and 24 have less resilience than the average resilience of shell 14 and a smaller thickness than the generally uniform thickness of shell 14. A user compresses bag 12 by squeezing compressible regions 18 and 20 towards each other. Since compressible regions 18 and 20 are softer and less resilient, it very difficult for the user to over inflate the patients lung when compression of bag 12 takes place via compressible regions 18 and 20. It is preferred that compressible regions are circular detents that are of sufficient size to allow for placement of an adult responder's finger tips.

The invention further includes a breathable facial mask 26 that is communicable with breathable gas chamber 16. Preferably, a breathable gas manometer 38 is connectable between a breathable gas outlet 28 of bag breathable gas chamber 16 and facial mask 26. A suitable breathable gas manometer for use with the present invention is disclosed in U.S. Pat. No. 5,557,049 to Ratner and is incorporated in its entirety herein.

Moreover, an oxygen supply line 32 is a breathable gas conduit between a medical oxygen source 30 and bag breathable gas chamber 16. An oxygen collection bladder 36 is communicable with a breathable gas inlet 34 through which breathable gas enters breathable gas chamber 16. Bladder 36 collects oxygen from medical gas source 30. It is preferred that breathable gas inlet 34 also be partially open to ambient air so that the pure oxygen collected by bladder 36 will be mixed with ambient air before being delivered to the patient during the compression of bag 12.

In operation of cardiopulmonary resuscitation device 10, a rescue person will first interface the patient with the bag by way of facemask 26. The rescuer will need to establish that facemask 26 is sealed onto the patient's face covering the patient's nose and mouth. The rescuer will then grasp the shell, placing his or her thumb and forefinger on thin walled compressible regions 18 and 20 and firmly squeeze the bag. Air will be expelled through breathable gas outlet 28 into facial mask 26. Once sufficiently compressed, the rescuer will release bag 12 to allow it to restore under its own resiliency, thereby pulling more breathable gas through shell inlet 34 and into breathable gas chamber 16. The rescuer will alternately compress and release shell 14 in like fashion at a normal breathing rate until the patient's breathing is restored or until advanced breathing equipment is made available to the patient. If bag 12 is optionally equipped with an oxygen source 30, line 32 and collection bladder 36, the rescuer will turn on oxygen source 30 to enrich the breathable gas oxygen content.

Certain modifications and improvements will occur to those skilled in the art upon a reading of the foregoing description. Such modifications and improvements have been deleted herein for the sake of conciseness and readability but are properly with the scope of the following claims.

What is claimed is:

1. A device to assist with cardio pulmonary resuscitation comprising:
a manually compressible and resiliently expandable bag constructed of an elastomer shell that encloses a breathable gas chamber having a volume capacity sufficient to inflate the lungs of a human patient when said bag is compressed, said shell having at least one compressible region constructed of a wall having less resilience than said shell's average resilience.

2. The cardiopulmonary resuscitation device of claim 1, wherein said at least one compressible region of lesser resilience is a circular detent.

3. The cardiopulmonary resuscitation device of claim 1, wherein said shell has an average shell thickness and said at least one compressible regions has an average wall thickness, wherein said average shell thickness is substantially greater than said wall thickness of said at least one compressible region.

4. The cardiopulmonary resuscitation device of claim 1, wherein said breathable gas chamber has a breathable gas outlet that is communicable with a breathable gas facial mask.

5. The cardiopulmonary resuscitation device of claim 1, wherein said breathable gas chamber has a breathable gas inlet that is communicable with ambient air.

6. The cardiopulmonary resuscitation device of claim 1, wherein said bag includes an oxygen supply line communicable with a medical oxygen source and said breathable gas chamber.

7. The cardiopulmonary resuscitation device of claim 6, further including a medical oxygen collection bladder communicable with said oxygen supply line and said breathable gas chamber.

8. The cardiopulmonary resuscitation device of claim 1, wherein said bag is molded using liquid silicone.

9. A device to assist with cardio pulmonary resuscitation comprising:
a manually compressible and resiliently expandable bag constructed of an elastomer shell that encloses a breathable gas chamber having a volume capacity sufficient to inflate the lungs of a human patient when said bag is compressed, said shell having an average shell thickness and at least one compressible region constructed of a wall having a wall thickness less than said average shell thickness.

10. The cardiopulmonary resuscitation device of claim 9, wherein said at least one compressible region is a circular detent.

11. The cardiopulmonary resuscitation device of claim 9 wherein said average shell thickness is substantially greater than said wall thickness of said at least one compressible region.

12. The cardiopulmonary resuscitation device of claim 9, wherein said breathable gas chamber has a breathable gas outlet that is communicable with a breathable gas facial mask.

13. The cardiopulmonary resuscitation device of claim 9, wherein said breathable gas chamber has a breathable gas inlet that is communicable with ambient air.

14. The cardiopulmonary resuscitation device of claim 9, wherein said bag includes an oxygen supply line communicable with a medical oxygen source and said breathable gas chamber.

15. The cardiopulmonary resuscitation device of claim 9, further including a medical oxygen collection bladder communicable with said oxygen supply line and said breathable gas chamber.

16. The cardiopulmonary resuscitation device of claim 9, wherein said bag is sized to define a breathable gas chamber with a volume capacity just sufficient to inflate the lungs of a human infant.

17. A cardiopulmonary resuscitation device comprising:
a) a manually compressible and resiliently expandable bag constructed of an elastomer shell that encloses a breathable gas chamber having a volume capacity sufficient to inflate the lungs of a human patient when said bag is compressed, said shell having at least one compressible region constructed of a wall having less resilience than said shell's average resilience;
b) a patient interface assembly that is communicable with a breathable gas outlet of said bag breathable gas chamber; and
c) a breathable gas source communicable with an inlet of said bag breathable gas chamber.

18. The cardiopulmonary resuscitation device of claim 17, wherein said bag has an average shell thickness and the at least one compressible region has a substantially thinner wall thickness.

19. The cardiopulmonary resuscitation device of claim 17, wherein said device includes an oxygen supply line communicable with a medical oxygen source and said breathable gas chamber.

20. The cardiopulmonary resuscitation device of claim 19, further including a medical oxygen collection bladder communicable with said oxygen supply line and said breathable gas chamber.

21. The cardiopulmonary resuscitation device of claim 17, further including a breathable gas manometer in communication with said breathable gas chamber for monitoring the pressure of breathable gas within said breathable gas chamber during compression.

22. The cardiopulmonary resuscitation device of claim 17, wherein said bag is sized to have a breathable gas chamber with a volume capacity just sufficient to inflate the lungs of a human infant.

23. The cardiopulmonary resuscitation device of claim 17, wherein said patient interface assembly is a breathable gas facial mask.

24. A cardiopulmonary resuscitation device for resuscitating human infants, said device comprising:
a) a manually compressible and resiliently expandable bag constructed of an elastomer shell that encloses a breathable gas chamber having a volume capacity sufficient to inflate the lungs of a human infant patient when said bag is compressed, said shell having at least one compressible region constructed of a wall having less resilience than said shell's average resilience, wherein said bag's average shell thickness is substantially greater than the wall thickness of said at least one compressible region of lesser resilience;
b) a breathable gas facial masked that is sized for human infants and is communicable with a breathable gas outlet of said bag breathable gas chamber; and
c) at least one breathable gas source communicable with an inlet of said bag breathable gas chamber.

25. The cardiopulmonary resuscitation device of claim 24, wherein at least one breathable gas source includes ambient air and a medical oxygen supply.

26. The cardiopulmonary resuscitation device of claim 25, further including a medical oxygen collection bladder communicable with said medical oxygen supply and said breathable gas chamber.

27. The cardiopulmonary resuscitation device of claim 26, further including a medical oxygen collection bladder communicable with said oxygen supply line and said breathable gas chamber.

28. The cardiopulmonary resuscitation bag device of claim 24, further including a breathable gas manometer in communication with said breathable gas chamber for monitoring the pressure of breathable gas within said breathable gas chamber during compression.
